(19) United States
(12) Patent Application Publication
(10) Pub. No.: US 2022/0274833 A1
(43) Pub. Date: Sep. 1, 2022

(54) PROCESS FOR MANUFACTURING AN AQUEOUS HYDROGEN PEROXIDE SOLUTION

(71) Applicant: SOLVAY SA, Brussels (BE)

(72) Inventor: Karol LORENT, Strépy-Bracquegnies (BE)

(21) Appl. No.: 17/637,470

(22) PCT Filed: Sep. 11, 2020
(86) PCT No.: PCT/EP2020/075489

§ 371 Date: Feb. 23, 2022

(30) Foreign Application Priority Data
Sep. 11, 2019 (EP) 19196602.7

(51) Int. Cl. C01B 15/023; C07C 253/16; C07C 67/14

(52) U.S. Cl. CPC C01B 15/023; C07C 253/16; C07C 67/14

(57) ABSTRACT

A process for manufacturing an aqueous hydrogen peroxide solution comprising the following steps:—hydrogenating a working solution which comprises an alkylanthraquinone and/or tetrahydroalkylanthraquinone and a mixture of a non-polar organic solvent and a polar organic solvent;—oxidizing the hydrogenated working solution to produce hydrogen peroxide; and—isolating the hydrogen peroxide, wherein the polar organic solvent is 5-methyl-2-isopropylcyclohexanecarbonitrile (C11F).

The present invention relates to a process for manufacturing an aqueous hydrogen peroxide solution using a specific polar organic solvent, and to a new method for synthesizing said specific polar organic solvent.

Hydrogen peroxide is one of the most important inorganic chemicals to be produced worldwide. Its industrial applications include textile, pulp and paper bleaching, organic synthesis (propylene oxide), the manufacture of inorganic chemicals and detergents, environmental and other applications.

Synthesis of hydrogen peroxide is predominantly achieved by using the Riedl-Pfleiderer process (originally disclosed in U.S. Pat. Nos. 2,158,525 and 2,215,883), also called anthraquinone loop process or AO (auto-oxidation) process.

This well-known cyclic process makes use typically of the auto-oxidation of at least one alkylanthrahydroquinone and/or of at least one tetrahydroalkylanthrahydroquinone, most often 2-alkylanthraquinone, to the corresponding alkylanthraquinone and/or tetrahydroalkylanthraquinone, which results in the production of hydrogen peroxide.

The first step of the AO process is the reduction in an organic solvent (generally a mixture of solvents) of the chosen quinone (alkylanthraquinone or tetrahydroalkylanthraquinone) into the corresponding hydroquinone (alkylanthrahydroquinone or tetrahydroalkylanthrahydroquinone) using hydrogen gas and a catalyst. The mixture of organic solvents, hydroquinone and quinone species (working solution, WS) is then separated from the catalyst and the hydroquinone is oxidized using oxygen, air or oxygen-enriched air thus regenerating the quinone with simultaneous formation of hydrogen peroxide. The organic solvent of choice is typically a mixture of two types of solvents, one being a good solvent of the quinone derivative (generally a non-polar solvent for instance a mixture of aromatic compounds) and the other being a good solvent of the hydroquinone derivative (generally a polar solvent for instance a long chain alcohol or an ester). Hydrogen peroxide is then typically extracted with water and recovered in the form of a crude aqueous hydrogen peroxide solution, and the quinone is returned to the hydrogenator to complete the loop.

The use of di-isobutyl-carbinol (DIBC) as polar solvent is namely described in Patent applications EP 529723, EP 965562 and EP 3052439 in the name of the Applicant. The use of a commercial mixture of aromatics sold under the brand Solvesso®-150 (CAS no. 64742-94-5) as non-polar solvent is also described in said patent applications. This mixture of aromatics is also known as Caromax, Shellsol, A150, Hydrosol, Indusol, Solvantar, Solvarex and others, depending on the supplier. It can advantageously be used in combination with sextate (methyl cyclohexyl acetate) as polar solvent (see namely U.S. Pat. No. 3,617,219).

Most of the AO processes use either 2-amylanthraquinone (AQ), 2-butylanthraquinone (BQ) or 2-ethylanthraquinone (EQ). Especially in the case of EQ, the productivity of the working solution is limited by the lack of solubility of the reduced form of ETQ (ETQH). It is namely so that EQ is largely and relatively quickly transformed in ETQ (the corresponding tetrahydroalkylanthraquinone) in the process. Practically, that ETQ is hydrogenated in ETQH to provide H2O2 after oxidation. The quantity of EQH produced is marginal in regards of ETQH. It means that the productivity of the process is directly proportional to the amount of ETQH produced. The reasoning is the same for a process working with AQ or BQ instead of EQ. The hydrogenated quinone solubility issue is known from prior art and some attempts were made to solve it. Namely co-pending PCT application EP2019/056761 to the Applicant, discloses the use of non-aromatic cyclic nitrile type solvents as polar solvent in the mixture, more specifically the use of cyclohexane carbonitriles, and especially substituted ones (in which the nitrile function is protected from chemical degradation).

Although some molecules of this kind are known, their market availability is currently only very limited and anyway too small to satisfy the needs of an industrial AO process. Besides, they are often synthesized starting from expensive and/or non-environmental friendly raw materials. Hence, it is an object of the present invention to provide a process for manufacturing an aqueous hydrogen peroxide solution which uses a solvent which is cheap and bio-sourced.

The present invention therefore concerns a process for manufacturing an aqueous hydrogen peroxide solution comprising the following steps:
hydrogenating a working solution which comprises an alkylanthraquinone and/or tetrahydroalkylanthraquinone and a mixture of a non-polar organic solvent and a polar organic solvent;
oxidizing the hydrogenated working solution to produce hydrogen peroxide; and
isolating the hydrogen peroxide,

wherein the polar organic solvent is a 5-methyl-2-isopropylcyclohexanecarbonitrile (C11F).

In the process of the invention, which preferably is a continuous process operated in loop, a working solution is used which is hence preferably circulated in a loop through the hydrogenation, oxidation and purification steps.

The term “alkylanthraquinone” is intended to denote a 9,10-anthraquinone substituted in position 1, 2 or 3 with at least one alkyl side chain of linear or branched aliphatic type comprising at least one carbon atom. Usually, these alkyl chains comprise less than 9 carbon atoms and, preferably, less than 6 carbon atoms. Examples of such alkylanthraquinones are ethylanthraquinones like 2-ethylanthraquinone (EQ), 2-isopropylanthraquinone, 2-sec- and 2-tert-butylanthraquinone (BQ), 1,3-, 2,3-, 1,4- and 2,7-dimethylanthraquinone, amylanthraquinones (AQ) like 2-iso- and 2-tert-amylanthraquinone and mixtures of these quinones.

The term “tetrahydroalkylanthraquinone ” is intended to denote the 9,10-tetrahydroquinones corresponding to the 9,10-alkylanthraquinones specified above. Hence, for EQ and AQ, they respectively are designated by ETQ and ATQ, their reduced forms (tetrahydroalkylanthrahydroquinones) being respectively ETQH and ATQH.

Preferably, an AQ or EQ is used, the latter being preferred. In order to be able to also solubilize the quinone, the polarity of the solvent mixture is preferably not too high. Hence, there is preferably at least 30 wt % of non-polar solvent in the organic solvents mixture, and more preferably at least 40 wt %. Generally, there is not more than 80 wt % of this non-polar solvent, preferably not more than 60 wt % of it in the organic solvents mixture.

The non-polar solvent preferably is an aromatic solvent or a mixture of aromatic solvents. Aromatic solvents are for instance selected from benzene, toluene, xylene, tert-butylbenzene, trimethylbenzene, tetramethylbenzene, naphthalene, methylnaphthalene mixtures of polyalkylated benzenes, and mixtures thereof. The commercially available aromatic hydrocarbon solvent of type 150 from the Solvesso® series (or equivalent from other supplier) gives good results. S-150 (Solvesso®-150; CAS no. 64742-94-5) is known as an aromatic solvent of high aromatics which offer high solvency and controlled evaporation characteristics that make them excellent for use in many industrial applications and in particular as process fluids. The Solvesso® aromatic hydro-carbons are available in three boiling ranges with varying volatility, e.g. with a distillation range of 165-181° C., of 182-207° C. or 232-295° C. They may be obtained also naphthalene reduced or as ultra-low naphthalene grades. Solvesso® 150 (S-150) is characterized as follows: distillation range of 182-207° C.; flash point of 64° C.; aromatic content of greater than 99% by wt; aniline point of 15° C.; density of 0.900 at 15° C.; and an evaporation rate (nButAc=100) of 5.3.

As explained above, the hydrogenation reaction takes place in the presence of a catalyst (like for instance the one object of WO 2015/049327 in the name of the Applicant) and as for instance described in WO 2010/139728 also in the name of the applicant (the content of both references being incorporated by reference in the present application). Typically, the hydrogenation is conducted at a temperature of at least 45° C. and preferably up to 120° C., more preferably up to 95° C. or even up to 80° C. only. Also typically, the hydrogenation is conducted at a pressure of from 0.2 to 5 bar. Hydrogen is typically fed into the vessel at a rate of from 650 to 750 normal m3 per ton of hydrogen peroxide to be produced.

The oxidation step may take place in a conventional manner as known for the AO-process. Typical oxidation reactors known for the anthraquinone cyclic process can be used for the oxidation. Bubble reactors, through which the oxygen-containing gas and the working solution are passed co-currently or counter-currently, are frequently used. The bubble reactors can be free from internal devices or preferably contain internal devices in the form of packing or sieve plates. Oxidation can be performed at a temperature in the range from 30 to 70° C., particularly at 40 to 60° C. Oxidation is normally performed with an excess of oxygen, so that preferably over 90%, particularly over 95%, of the alkyl anthrahydroquinones contained in the working solution in hydroquinone form are converted to the quinone form.

After the oxidation, during the purification step, the hydrogen peroxide formed is separated from the working solution generally by means of an extraction step, for example using water, the hydrogen peroxide being recovered in the form of a crude aqueous hydrogen peroxide solution. The working solution leaving the extraction step is then recycled into the hydrogenation step in order to recommence the hydrogen peroxide production cycle, eventually after having been treated/regenerated.

In a preferred embodiment, after its extraction, the crude aqueous hydrogen peroxide solution is washed several times i.e. at least two times consecutively or even more times as required to reduce the content of impurities at a desired level.

The term “washing” is intended to denote any treatment, which is well known in the chemical industry (as disclosed in GB841323A, 1956 (Laporte), for instance), of a crude aqueous hydrogen peroxide solution with an organic solvent which is intended to reduce the content of impurities in the aqueous hydrogen peroxide solution. This washing can consist, for example, in extracting impurities in the crude aqueous hydrogen peroxide solution by means of an organic solvent in apparatuses such as centrifugal extractors or liquid/liquid extraction columns, for example, operating counter-current wise. Liquid/liquid extraction columns are preferred. Among the liquid/liquid extraction columns, columns with random or structured packing (like Pall rings for instance) or perforated plates are preferred. The former are especially preferred.

In a preferred embodiment, a chelating agent can be added to the washing solvent in order to reduce the content of given metals. For instance, an organophosphorus chelating agent can be added to the organic solvent as described in the above captioned patent application EP 3052439 in the name of the Applicant, the content of which is incorporated by reference in the present application.

The expression “crude aqueous hydrogen peroxide solution” is intended to denote the solutions obtained directly from a hydrogen peroxide synthesis step or from a hydrogen peroxide extraction step or from a storage unit. The crude aqueous hydrogen peroxide solution can have undergone one or more treatments to separate out impurities prior to the washing operation according to the process of the invention. It typically has an H2O2 concentration within the range of 30-50% by weight.

The solvents of the invention make it is possible to achieve a higher solubility and thus there is less polar solvent needed to achieve a higher partition coefficient. With this higher partition coefficient it is possible to reduce the capex (capital expenditure) required for the extraction sector. The solvents of the invention are particularly suitable for the manufacture of hydrogen peroxide by the AO-process wherein said process has a production capacity of hydrogen peroxide of up to 100 kilo tons per year (ktpa). Preferably said process is a small to medium scale AO-process operated with a production capacity of hydrogen peroxide of up to 50 kilo tons per year (ktpa), and more preferably with a production capacity of hydrogen peroxide of up to 35 kilo tons per year (ktpa), and in particular a production capacity of hydrogen peroxide of up to 20 kilo tons per year (ktpa). The dimension ktpa (kilo tons per annum) relates to metric tons.

A particular advantage of such a small to medium scale AO-process is that the hydrogen peroxide can be manufactured in a plant that may be located at any, even remote, industrial end user site and the solvents of the invention are therefore especially suitable. It is namely so that since their partition coefficient is more favourable, less emulsion is observed in the process and a purer H2O2 solution can be obtained (namely containing less TOC) and this for a longer period of time compared to when solvents known from prior art are used. In a preferred sub-embodiment of the invention, the working solution is regenerated either continuously or intermittently, based on the results of a quality control, regeneration meaning conversion of certain degradates, like epoxy or anthrone derivatives, back into useful quinones. Here also, the solvents of the invention are favourable because the quality of the H2O2 solution can be maintained within the specifications namely in terms of TOC for a longer period of time.

As explained above, the main feature of the invention is the recourse to a mixture of a polar organic solvent and a non-polar organic solvent wherein the polar organic solvent is C11F. This compound (5-methyl-2-isopropylcyclohexanecarbonitrile or C11F) has namely been synthesized starting from menthol by Debra K. Dillner (2009), Syntheses of C-1 Axial Derivatives of 1-Menthol, Organic Preparations and Procedures International, 41:2, 147-152, DOI:10.1080/00304940902802008.

In the method described in this paper, menthol was first reacted with methanesulfonyl chloride (mesyl chloride) in dichloromethane (DCM) with the addition of triethylamine (to trap the HCl generated) and then, the mesylate so obtained was reacted with KCN in acetonitrile and in the presence of 18-crown-6 (a phase transfer agent—which complexes the K ion and improves the solubility of KCN in the organic phase and enhance the nucleophile strength of formula [C2H4O]6) to generate the compound C11F. This paper also makes reference to a previous method starting from menthyl tosylate with NaCN in DMSO.

Hence, in a first embodiment, the C11F used in the process of the invention has been obtained by reaction of menthol with mesyl or tosyl chloride followed by the cyanation of the obtained mesylate or tosylate, preferably with KCN and/or NaCN.

This synthesis method has the drawback that organic reactives are used, which generate organic effluents.

Hence, in a second embodiment, the C11F used in the process of the invention has been obtained by reaction of menthol with phosphorus tribromide (PBr3), phosphorus trichloride (PCl3), phosphorus triiodide (PI3), potassium iodide (KI) with acid catalysis, thionyl chloride (SOCl2) or thionyl bromide (SOBr2), followed by the cyanation of the obtained bromide, iodide or chloride, preferably with KCN and/or NaCN.

Although these methods work in practice, they might be improved by the use of other reactives including much more efficient reactive groups which hence imply shorter reaction times. Hence, in a third preferred embodiment, the C11F used in the process of the invention has been obtained by reaction of menthol with an anhydride, acid or acyl chloride bearing a trifluoromethyl group, followed by cyanation.

Since this synthesis route has never been reported up to date, the present invention also relates to a method of manufacturing 5-methyl-2-isopropylcyclohexanecarbonitrile or C11F by an esterification reaction of menthol with an anhydride, a carboxylic acid or an acyl chloride bearing a trifluoromethyl group, followed by cyanation, preferably with KCN and/or NaCN.

The preferred reactives for the esterification reaction with menthol are TFAC (TriFluoroAcetylChloride), trifluoroacetic acid, trifluoromethanesulfonyl (triflic) anhydride or trifluoromethyl acetic anhydride. The esterification reaction medium preferably comprises a solvent for the menthol, like for instance dichloromethane (DCM), or any other inert aromatic solvent like toluene, or aliphatic solvent like alkane. In the case of TFAC or of other acyl chlorides, the esterification reaction medium preferably also comprises a compound able to trap the acid released (HCl) like pyridine, triethylamine, DIPEA (Hunig's base), proton sponge, imidazole, or any aromatics containing a pyridine-like nitrogen able to react with HCl to give the corresponding chlorhydrate salt, inorganic bases like Na2CO3, sodium bicarbonate etc. The esterification reaction preferably takes place at a temperature from −20 to 50° C., preferably at ambient temperature. It also preferably takes place at atmospheric pressure. In the case of TFAC, which is a gas, said TFAC can either be bubbling through the reaction mixture at atmospheric pressure, or the reaction can take place in an autoclave at a pressure up to 10 bar.

The anhydride, acid or acyl chloride used in the esterification reaction is preferably recovered, preferably by distillation or selective extraction.

As to the cyanation, it generally involves the use of compounds like KCN, NaCN and the like. KCN and/or NaCN are preferred for an industrial process mainly for economic reasons. Cyanation preferably takes place in a polar solvent like DMF, DMSO or sulfolane. The reaction temperature preferably is from 50 to 150° C., preferably between 100 and 140° C., most preferably about 120° C. The reaction generally happens at a pressure from atmospheric pressure up till 10 bar, mots preferably at atmospheric pressure and until full conversion is reached.

The present invention also relates to a method of manufacturing 5-methyl-2-isopropylcyclohexanecarbonitrile or C11F by reaction of menthol with phosphorus tribromide (PBr3), phosphorus trichloride (PCl3), phosphorus triiodide (PI3), potassium iodide (KI) with acid catalysis, thionyl chloride (SOCl2) or thionyl bromide (SOBr2), followed by the cyanation of the obtained bromide, iodide or chloride, preferably with KCN and/or NaCN. This method has also never been reported in literature.

1.-14. (canceled)

15. A process for manufacturing an aqueous hydrogen peroxide solution comprising the following steps:
hydrogenating a working solution which comprises an alkylanthraquinone and/or tetrahydroalkylanthraquinone and a mixture of a non-polar organic solvent and a polar organic solvent;
oxidizing the hydrogenated working solution to produce hydrogen peroxide; and
isolating the hydrogen peroxide, wherein the polar organic solvent is 5-methyl-2-isopropylcyclohexanecarbonitrile (C11F).

16. The process according to claim 15, said process having a production capacity of hydrogen peroxide of up to 100 kilo tons per year.

17. The process according to claim 15, said process being operated in a plant located at an industrial end user site.

18. The process according to claim 15, wherein the C11F has been obtained by reaction of menthol with mesyl or tosyl chloride followed by cyanation.

19. The process according to claim 15, wherein the C11F has been obtained by reaction of menthol with phosphorus tribromide (PBr3), phosphorus trichloride (PCl3), phosphorus triiodide (PI3), potassium iodide (KI) with acid catalysis, thionyl chloride (SOC12) or thionyl bromide (SOBr2), followed by cyanation.

20. The process according to claim 15, wherein the C11F has been obtained by an esterification reaction of menthol with an anhydride, an acid or an acyl chloride said anhydride, acid or acyl chloride bearing a trifluoromethyl group, followed by cyanation.

21. A method of manufacturing 5-methyl-2-isopropylcyclohexanecarbonitrile by an esterification reaction of menthol with an anhydride, acid or acyl chloride said anhydride, acid or acyl chloride bearing a trifluoromethyl group, followed by cyanation.

22. The method according to claim 21, wherein the esterification reaction uses TFAC (TriFluoroAcetylChloride), trifluoroacetic acid, triflic anhydride or trifluoromethyl acetic anhydride.

23. The method according to claim 21, wherein the esterification reaction medium comprises a solvent for the menthol.

24. The method according to claim 21, wherein the esterification reaction uses an acyl chloride and wherein the esterification reaction medium comprises a compound able to trap the acid released (HCl) like pyridine, triethylamine, DIPEA (Hunig's base), proton sponge, imidazole, any aromatic molecule containing a pyridine-like nitrogen able to react with HCl to give the corresponding chlorhydrate salt, inorganic bases.

25. The method according to claim 21, wherein the esterification reaction uses TFAC and either said TFAC is bubbling through the reaction mixture at atmospheric pressure, or the esterification reaction takes place in an autoclave at a pressure up to 10 bar.

26. The method according to claim 21, wherein the anhydride, acid or acyl chloride is recovered by distillation or selective extraction.

27. The method according to claim 21, wherein the cyanation involves the use of KCN and/or NaCN.

28. The method according to claim 27 wherein the cyanation takes place in a polar solvent.

29. The method of claim 28 wherein the polar solvent is selected from the group consisting of DMF, DMSO and sulfolane.

30. A method of manufacturing 5-methyl-2-isopropylcyclohexanecarbonitrile by reaction of menthol with phosphorus tribromide (PBr3), phosphorus trichloride (PCl3), phosphorus triiodide (PI3), potassium iodide (KI) with acid catalysis, thionyl chloride (SOCl2) or thionyl bromide (SOBr2), followed by cyanation.
